(19) United States
(12) Patent Application Publication
(10) Pub. No.: US 2013/0052255 A1
(43) Pub. Date: Feb. 28, 2013

(54) NAIL DISCOLORATION AND FUNGUS TREATMENT

(71) Applicants: Karen Carmody Smith, Lorena, TX (US); Sameer Shums, Waco, TX (US)

(72) Inventors: Karen Carmody Smith, Lorena, TX (US); Sameer Shums, Waco, TX (US)

(73) Assignee: Spenco Medical Corporation, Waco, TX (US)

(21) Appl. No.: 13/695,398

(22) PCT Filed: May 3, 2011
(86) PCT No.: PCT/US11/35048

§ 371 Date: Oct. 30, 2012

Related U.S. Application Data
(60) Provisional application No. 61/330,821, filed on May 3, 2010.
(60) Provisional application No. 61/330,702, filed on May 3, 2010.

(51) Int. Cl. A61K 33/38; A61K 9/14; A61K 9/70; A61K 9/00; A61P 31/10; A61K 33/00; A61K 33/34; B82Y 5/00

(52) U.S. Cl. 424/443; 424/600; 424/618; 424/400; 424/630; 977/915; 977/810; 977/773

(57) ABSTRACT

A system and method for treating human nails afflicted with fungus, discoloration, or which are unusually thick is now disclosed. The system comprises a hydrogel moist pad or pad, a treatment agent comprising a metallic colloidal suspension having antifungal activity in an aqueous environment, and an adhesive for securing the hydrogel and treatment agent to a nail in need of such treatment. A method and regimen for treatment of the nail is also disclosed.

### CROSS REFERENCE TO RELATED APPLICATIONS

This application claims the benefit of U.S. Provisional Patent Application Ser. Nos. 61/330,702 filed on May 3, 2010 and 61/330,821 filed on May 3, 2010.

### TECHNICAL FIELD OF INVENTION

This invention relates to the field of systems and methods for treating humans having toenails or fingernails afflicted with fungus and/or which are discolored or unusually thickened.

### BACKGROUND OF THE INVENTION

Nail fungus (also called onychomycosis of the nail plate or tinea of the nails) is a difficult condition to eradicate. In this condition, which may be of concern primarily to human patients, fungus grows under the nail plate which ironically provides a protective covering for the fungus and makes it difficult for medicaments to attack the fungus. The condition may affect both toenails and fingernails, although it is more prevalent in toenails since the wearing of shoes provides a dark, moist environment favored by fungal organisms.

Various systematic and topical treatment agents have been reported for the treatment of nail fungus. Systematic agents, pharmaceutical compositions taken orally by a patient, may be contraindicated for certain patients and/or may require many months of treatment. These agents may adversely affect the liver and some of these systemic medications require testing of the liver during the treatment process. In addition, patients may be allergic to oral antifungal agents.

Topical agents which would otherwise be effective against fungus have suffered from the inability to reach the fungus under the protective nail.

Nails may also be cosmetically unattractive due to fungus or other etiologies and appear discolored or unusually thickened.

### DETAILED DESCRIPTION OF THE INVENTION

A system and method for treatment of fungus-afflicted nails, discolored nails, and abnormally thickened nails is now disclosed. The system is preferably provided as a kit having a hydrogel moist pad, a topical agent having a component dispersed in a carrier, and a covering adapted for securing the hydrogel moist pad and topical agent in place for the treatment period.

A preferred hydrogel moist pad comprises cross-linked polyethylene oxide and water in a moist pad format. A suitable hydrogel formulation is disclosed in U.S. Pat. No. 3,410,006 to King, which is herein incorporated by reference. The hydrogel moist pad provides moisture to the area to which it is applied. Upon application of a moist pad to the skin and/or nail of a user, the moist pad provides a moist environment. Most preferably, a hydrogel moist pad, consisting of cross-linked polyethylene oxide, and containing up to 96% water, supported upon a net of low density polyethylene is employed. The hydrogel will dry out upon prolonged exposure to air.

The moist nature of the hydrogel provides moisture to the nail being treated during use. The nail becomes softened and penetrable, and the moisture activates the fungus. This activation allows dormant fungus under the nails to be made ready for uptake of a treatment agent provided concurrently with the hydrogel to the nail. The preferred treatment agent is active in an aqueous environment and upon uptake by the active fungus, eradicates the organism.

Because some of the fungus spores may remain dormant during a first treatment period, in a preferred embodiment, the regimen further comprises a rest period after conclusion of the first treatment period. This rest period is then followed by a second treatment period. This allows the previously dormant spores to activate due to hydration, and for uptake of the treatment agent provided concurrently with the hydrogel to the nail. In severe cases, additional periods of rest followed by treatment periods can be applied. It is believed that this regimen will eradicate the fungus when one-time treatments may not.

A preferred polyethylene oxide is Union Carbide POLYOX WSR-1105. The moist pad may be made by introducing a polymer (solid) into water, creating a feed mix. The feed mix is used to coat a web material (or scrim) and two outer linings are applied creating sheets of hydrogel. These sheets are then introduced to a high energy field which cross-links the polymers giving the hydrogel greater molecular integrity and thereby creating sheets of hydrogel. Cross linking may be accomplished through chemical cross linking known to the art. Utilizing an electron beam accelerator is preferred. Electron beam cross-linking is achieved through the introduction of the high energy field, created by the accelerated electrons, which causes the release of hydrogen atoms thereby causing carbon molecule covalent bonding. The creation of longer chains of the polymer in the gel increases its molecular integrity, giving the gel the desired characteristics. The benefits of electron beam cross-linking include: precise control of the amount of polymer cross-linking; other types allow for the continuation of cross-linking over a period of time, the ability to eliminate the need for chemical cross-linking agents which may complicate or interfere with other additives or active ingredients; and the ability to manufacture high quality hydrogels on a consistent basis.

The physical characteristics can be further modified by varying the percent of polymer cross-linking and the way in which the high energy field is delivered. There are three variables in the use of an electron beam accelerator for cross-linking of hydrogels, including time of exposure of the target material to the electron stream, the voltage (electrical potential); and amperage (strength of the electrical current).

A preferred hydrogel moist pad suitable for the system and method of the invention is commercially available from Spenco Medical Corporation (Waco, Tex.) under the trademark 2nd Skin®. 2nd Skin® hydrogel moist pads are provided as a hydrogel moist pad sandwiched between a first sheet of polyethylene film (clear) and a second sheet of polyethylene film (blue). For use in this system and method, one sheet of polyethylene film is removed prior to application, exposing the moist hydrogel which is applied to the affected nail as further described below.

A topical treatment agent useful in the system of the invention is a metallic colloidal suspension, wherein said metallic component has antifungal activity in an aqueous environment.

The topical treatment agent of the invention can be selected from bioactive silvers which have antifungal killing activity. To test for appropriate antifungal activity for use in the invention, a known quantity of fungal agent in CFU/ml can be treated with a candidate for use. The candidate may be tested for its fungicidal activity against common fungus which afflicts toenails such as Trichophyton rubrum. A known quantity of organism (CFU/mL) can be placed in a standard broth medium. After treatment with the candidate for use, testing of CFU/mL for one or more time points up to 24 hours is done. It is preferred to use a topical agent in a concentration which will provide a significant reduction in colony forming units (>2 log reduction) in 24 hours or that 95%-100% of the organism is eradicated in 24 hours.

Further properties of the metallic colloidal may be nontoxic to humans and nonsensitizing in low concentrations and/or does not result in significant irritation or sensitivities at the concentration used.

Fine or ultrafine silver particles, such as colloidal suspensions of a high content (96%) or ultra-fine, positively charged silver ions in purified water are useful topical treatment agents. A suitable product is available from Natural-Immunogenics Corporation under as SOVEREIGN SILVER®, which is a suspension of particles less than 1 nm in size. The average particle size is 0.8 nm and the concentration is about 10 ppm as supplied. Another suitable product is Argentyn 23®, also available from Natural Immunogenics Corporation. It contains 96% actively charged silver particles (positively charged) and has a particle size of about 0.8 nanometers. It has a concentration of about 23 ppm as supplied.

A suitable concentration for use in a method for treatment is from about 10 ppm to about 23 ppm.

Another suitable topical treatment agent may be selected from positively charged silver nanoparticles from 1 to 100 nanometers at a concentration from 10 to 50 ppm. These nanoparticles are distinguished from those described in U.S. patents made according to U.S. Pat. No. 7,135,195, 6,743,348 or 6,214,299 and includes all other positively charged silver nanoparticles made by other methodologies. Nanosilver particles can be made in various ways. The most common methods are using sodium borohydride or sodium citrate as reducing agents and silver nitrate as the metal salt precursor in an aqueous solvent. Citrate or polyvinylpyrrolidone (PVP) might be required as stabilizing agents. (Krutyaknov, et al, Russian Chemical Reviews, 77(3) 233-257).

In an alternate embodiment, said metallic colloidal suspension, is a metallic copper hydrosol, preferably comprised of particles less than 100 nanometers in size, and preferably having an average particle size between about 0.5 and 100 nanometers.

The topical treatment agent of the invention is a metallic colloidal suspension wherein the metallic particles are suspended in a pharmaceutical carrier suitable for topical application. Most preferably, the carrier is a gel. A preferred gel is an amorphous water-based gel. Moisture is managed using an aqueous base combined with a blend of hydrophilic substances. A most preferred gel comprises purified water, triethanolamine (TEA) and carbomer.

In a preferred regimen for use of the system of the invention, a user may first prepare the nail for treatment. This may include trimming the nail afflicted with fungus and scraping debris from under the nail. After cleaning the area, or after a shower or bath, the topical agent having a metallic colloidal suspension component, wherein said metallic component has antifungal activity in an aqueous environment in a carrier is applied to the surface of the nail and the skin at the base of the nail as much as possible.

The nail and topical treatment agent will then be covered with a hydrogel moist pad. The hydrogel moist pad can be customized for the user so that it covers the entire surface of the nail, most preferably over the top of the nail and also over the topical agent pushed under the nail. In another embodiment, pre-cut moist pad portions adapted for different sizes of fingernails or toenails can be supplied.

Ideally, the hydrogel moist pad should overlap only about 2 mm on the skin surrounding the nail to avoid prolonged hydration of the sides and bottom of the toe.

If covered with a protective film as supplied, the protective film will be removed from one side of the moist pad to expose the hydrogel. The exposed moist hydrogel is placed on the nail to which the topical agent has already been applied. The other layer of the protective film is preferably left in place on the hydrogel moist pad. The protective film left in place will assist in keeping the moisture in the hydrogel moist pad and avoiding desiccation.

In order to secure the hydrogel moist pad and topical treatment agent to the nail, in a preferred embodiment a kit is supplied, that, in addition to a hydrogel moist pad and a topical treatment agent, contains a bandage or tape for application to the finger or toe and nail. A bandage can be pre-cut in a shape adapted for a human digit. Some suitable shapes for such a bandage are “T-shaped” and “L-shaped.” A T-shaped bandage is disclosed in U.S. Pat. No. 2,440,235 to M. Solomon which is herein incorporated by reference. An example of an “L-shaped” bandage is disclosed in U.S. Pat. No. 3,880,159 to Diamond which is herein incorporated by reference.

It is desirable that the bandage have a tab which may be folded over the tip and nail of the digit. For example, when an L shaped adhesive bandage which will function as an occlusive or semi-occlusive covering, is employed, the shorter end of the L is wrapped over the tip of the nail covering the clear film of the hydrogel which overlies the topical treatment agent. The edges of the adhesive are then sealed to hold in moisture. The longer end will wrap around the digit and secure the hydrogel moist pad in place. In the case of a T-shaped bandage, a first leg of the T may be folded over the tip and nail of the finger or toe and the other two legs wrapped around the digit to secure said first leg, as illustrated in U.S. Pat. No. 2,440,235, FIG. 4. In the case of the instant invention, the topical agent will first be applied to the nail, followed by a hydrogel moist pad, and then the first leg of the T folded over the hydrogel moist pad. The other two legs are then folded over the first leg to further secure it in place. Another bandage which may be used comprises two strips of adhesive that can be crossed over in application. In such case, the first strip of adhesive is placed over the film of the hydrogel moist pad. It is secured around the toe and adhesively secured to the underside of toe. A second strip of adhesive is preferably placed over the length of the toe. It should be perpendicular to the first tape and is secured underneath the tip of the toe at one end and also on top of the foot to completely seal in the topical agent.

Alternatively, other pre-shaped bandages suitable to seal the topical agent covered with the hydrogel moist pad can be utilized.

The bandage is preferably made of a material which will secure the topical agent covered with the hydrogel moist pad in place on the nail and will prevent or slow desiccation of the hydrogel moist pad. A moisture impermeable flexible film or fabric is preferred. In a preferred embodiment, the bandage is made of a spun lace polyester non-woven fabric, laminated with an adhesive coating and release paper, and pre-cut into a desired shape. Upon use, the release paper is removed which exposes the adhesive coating which is then applied to the hydrogel moist pad/topical agent assembly on the nail of the human digit, thereby securing the assembly in place providing a seal to prevent moisture loss.

In one regimen, the treatment is left in place for approximately 24 hours then removed. The area is then cleaned and a second application applied. This is repeated every 24 hours for three to ten consecutive days. Preferably, the treatment is conducted for at least seven days and most preferably ten days.

The treatment area is then left untreated for at least several days and preferably up to three weeks.

Treatment with the topical agent and hydrogel moist pad is then preferably resumed for three to ten consecutive days according to the procedure for the first treatment.

The treatment and waiting period is then repeated for one to four, preferably one to six cycles, or until the toenail is healthy and free from apparent fungal affliction and/or discoloration or abnormal thickening.

Without wishing to be bound to any theory, it is believed that a metallic colloidal suspension in which the nano-sized charged metal is dispersed in water and held in suspension acts in concert with the hydrogel moist pad, which has a high water content, to disperse the nanoparticles of metal to the tissues.

The topical treatment agent is active in an aqueous environment which is maintained by the hydrogel moist pad placed over it. It is believed that the hydrogel moist pad also acts to soften the nail, thereby increasing its penetrability, and allows delivery of the antifungal topical treatment agent to the area underneath the nail which has previously only been successfully achieved consistently with systemic therapy.

The water in the hydrogel and the moist environment provided by the hydrogel and maintained by the occlusive or semi-occlusive moist pad, helps to activate the fungal spores so that they can be eliminated. Dormant spores are very difficult to eradicate until they become active.

In an alternative embodiment, a hydrogel moist pad is impregnated with the topical treatment agent consisting of a metallic colloidal suspension and the impregnated moist pad is applied to a nail to treat fungus, discoloration, or unusual nail thickening. An adhesive bandage is preferably employed to keep the impregnated hydrogel moist pad in place on the nail during treatment.

In still another embodiment, a treatment system is supplied which comprises a hydrogel moist pad having a border edge, an adhesive secured to said hydrogel moist pad and extending outwardly from said border edge. The portion of said adhesive which extends outwardly is secured to release paper as supplied. In use, the release paper is removed and the adhesive is exposed and applied over the hydrogel moist pad to secure the hydrogel moist pad to the affected area. In this embodiment, the hydrogel moist pad defines an indentation therein, said indentation pre-loaded with a metallic colloidal treatment agent. Alternatively, the hydrogel moist pad is impregnated with metallic colloidal treatment agent. The treatment system can be applied to the area to be treated in one step. In still another embodiment, the metallic colloidal treatment agent may be delivered to the area from an impregnated woven or non-woven material which can be placed on the nail and contacted by the hydrogel moist pad. The moisture in the hydrogel moist pad causes the treatment agent to be released from the material.

Kill Study with Sovereign Silver® Colloidal Silver

TABLE 1

| | Organism | |
|---|---|---|
| | Trichophyton mentagrophytes | Trichophyton rubrum |
| | ATCC 9533 | ATCC 28188 |
| | CFU/1 mL | CFU/1 mL |
| Organism Count | 4.9 × 10⁶ | 9.6 × 10⁶ |
| Initial Count | 5.4 × 10⁶ | 9.5 × 10⁶ |
| 5 Minute Count | 1.0 × 10⁴ | 1.1 × 10⁵ |
| 15 Minute Count | 8.3 × 10² | 1.3 × 10⁴ |
| 1 Hour Count | 1.1 × 10² | 8.4 × 10² |
| 24 Hour Count | 0.5 × 10¹ | 0.5 × 10¹ |
| 48 Hour Count | 0 | 0 |
| 7 Day Count | 0 | 0 |
| 30 Day Count | 0 | 0 |

### Example 1

A human patient presents with a discolored thickened right great toenail consistent with severe onychomycosis. The patient's nail is trimmed, debris is cleaned out from under the nail and the skin cleaned. The topical treatment agent in a gel is applied to the top of the nail and under the tip of the nail. A hydrogel square (2nd Skin® hydrogel bandage, available from Spenco Medical Corporation, Waco, Tex.) is applied over the topical treatment agent and secured to the toe with an occlusive adhesive. This process is repeated daily for 7 to 10 days followed by a three week rest. The nail is observed and the regiment repeated if necessary to completely eradicate fungus from the nail.

### Example 2

A person suffers from unusually thickened toenails. They trim their nail, clean any debris from under the nail, and wash the toes thoroughly. A hydrogel square (2nd Skin® hydrogel bandage, available from Spenco Medical Corporation, Waco, Tex.), which has been pre-infused with colloidal metallic copper as described above, is placed directly on the thickened toenail and secured to the toe with an occlusive adhesive. This process is repeated daily for 7 to 10 days followed by a three week rest and 7 to 10 days additional treatment to complete a cycle of treatment. One or more additional cycles of treatment may be undertaken until the nail has healthy growth.

We claim:

1. A system for treatment of nails afflicted with fungus, discoloration or unusual thickening, comprising a topical treatment agent consisting of a metallic colloidal suspension, wherein said metallic colloidal suspension has fungicidal activity in an aqueous environment, a hydrogel moist pad, and a means for securing said topical agent covered by said hydrogel moist pad to the nail of a person in need of such treatment.

2. The system of claim 1, wherein said hydrogel moist pad comprises cross-linked polyethylene oxide and water.

3. The system of claim 2, wherein said polyethylene oxide is cross linked utilizing an electron beam accelerator.

4. The system of claim 1 wherein said metallic colloidal suspension is a positively charged colloidal silver hydrosol having an average particle size of less than 1 nanometer.

5. The system of claim 1, wherein said metallic colloidal suspension is a positively charged colloidal silver having an average particle size from about 1 nm to about 100 nm.

6. The system of claim 1, wherein said metallic colloidal suspension is a metallic copper hydrosol.

7. The system of claim 6, wherein said metallic copper hydrosol comprises particles having an average particle size between about 0.5 and 100 nanometers.

8. The system of any of the above claims, wherein a topical pharmaceutical carrier is admixed with said metallic colloidal agent and said agent is dispersed therein.

9. A method for treatment of nails afflicted with fungus, discoloration or unusual thickening, comprising applying a topical treatment agent consisting of a metallic colloidal suspension, wherein said metallic component has antifungal activity in an aqueous environment to an afflicted nail, covering the topical agent with a hydrogel moist pad, securing said agent covered with said hydrogel to said nail for an appropriate interval and repeating one or more times.

10. The method of claim 9 wherein said metallic colloidal suspension is a positively charged colloidal silver hydrosol having an average particle size of less than 1 nanometer.

11. The method of claim 9, wherein said metallic colloidal suspension is a positively charged colloidal silver having an average particle size from about 1 nm to about 100 nm.

12. The method of claim 9, wherein said metallic colloidal suspension is a metallic copper hydrosol.

13. The method of claim 12, wherein said metallic copper hydrosol comprises particles having an average particle size between about 0.5 and 100 nanometers.

14. The method of claim 9, wherein a topical pharmaceutical carrier is admixed with said metallic colloidal suspension and said metallic colloidal suspension is dispersed therein.

15. The method of claim 14, wherein said topical pharmaceutical carrier is a water-based gel.

16. The method of claim 9, wherein said metallic colloid is impregnated into said hydrogel moist pad.

17. The method of claim 9, wherein said metallic colloid is impregnated into a substrate and said substrate releases said metallic colloid upon hydration from said hydrogel moist pad.

18. The method of claim 9, wherein said treatment is repeated for a first period ranging from three to ten days.

19. The method of claim 18, wherein after said first period, no treatment is applied for a second period, and treatment is applied for a third period.

20. The method of claim 19, wherein said first period is three to ten days.

21. The method of claim 19, wherein said second period is one to three weeks.

22. The method of claim 19, wherein said third period is three to ten days.

23. The method of claim 19, wherein said first, second and third periods together constitute a cycle, and one to six cycles are undertaken to treat said nails.

24. The method of claim 23, wherein three to six cycles are undertaken to treat said nails.

25. The method of claim 23, wherein each cycle takes approximately one month.

26. The method of claim 9, wherein said interval is 24 to 72 hours.

27. The method of claim 9, wherein said interval is 24 hours.

28. A kit for treating human nails, selected from fingernails or toenails, on a patient in need of treatment for discolored or thickened nails, comprising:
a. sufficient metallic colloidal treatment agent to treat human nails in need of such treatment for a pre-selected period of time;
b. one or more hydrogel moist pads in packages suitable for preventing desiccation of said pads; and
c. instructions for using said treatment agent and said hydrogel moist pads in conjunction with one another, which instructions specify that said treatment agent when removed from said one or more containers is to be placed in direct contact said with nails in need of such treatment, and said hydrogel moist pad when removed from said packages is made to directly contact said treatment agent.

29. The kit of claim 28, wherein said treatment agent is a metallic colloid in a pharmaceutical carrier;

30. The kit of claim 28, wherein said treatment agent is a metallic colloid impregnated into a substrate which metallic colloid is releasable from said substrate upon hydration.

31. The kit of claim 28, wherein said metallic colloid is impregnated into said hydrogel moist pad.

32. The kit of claim 28, further comprising an occlusive bandage suitable for securing said nanosilver treatment agent and said hydrogel moist pad to a finger or toe to which said nail is appended.

33. The kit of claim 28, wherein said instructions comprise an instruction that a fresh application of a combination of treatment agent and hydrogel moist pad is to be applied to said nail daily for a period of three to ten days.
